(19) United States
(12) Patent Application Publication
(10) Pub. No.: US 2010/0174153 A1
(43) Pub. Date: Jul. 8, 2010

(54) METHOD, APPARATUS AND PROGRAM FOR EVALUATING LIFE STYLES

(75) Inventor: Kazuhiro Nakagawa, Saitama (JP)

Correspondence Address: K&L Gates LLP, P. O. BOX 1135, CHICAGO, IL, 60690, US

(73) Assignee: Sony Corporation, Tokyo (JP)

(21) Appl. No.: 12/651,845

(22) Filed: Jan. 4, 2010

(30) Foreign Application Priority Data
Jan. 6, 2009 (JP) 2009-000545

(51) Int. Cl. A61B 5/024; G06F 17/10; A61B 5/00; A61B 5/11

(52) U.S. Cl. 600/301; 703/2; 600/300; 600/595; 600/508

(57) ABSTRACT

Disclosed herein is a life-style evaluation method including the steps of acquiring information on a form of a living habit prescribing a life style, obtaining a virtual biological-clock rhythm curve by reflecting the information in a reference biological clock rhythm curve on a basis of the degree of an effect given by the form of the living habit on a biological-clock rhythm curve, and setting an indicator of the life style on a basis of the virtual biological-clock rhythm curve.

### CROSS REFERENCES TO RELATED APPLICATIONS

The present application that claims priority to Japanese Patent Application No. 2009-000545 filed in the Japanese Patent Office on Jan. 6, 2009, the entire contents of which are incorporated herein by reference.

### BACKGROUND

In general, the present application relates to a life-style evaluation method for evaluating a life style, an information processing apparatus for implementing the life-style evaluation method and a life-style evaluation program to be executed by the information processing apparatus to implement the life-style evaluation method. To put it in detail, the present application relates to a life-style evaluation method for evaluating a variety of life styles prescribed by a variety of living habits, an information processing apparatus for implementing the life-style evaluation method and a life-style evaluation program to be executed by the information processing apparatus to implement the life-style evaluation method.

In the past, people lived in society by practicing similar life styles. That is to say, people get up at about the same time, go to bed at about the same time, eat about the same meals at about the same times and work in about the same period of time. In this modern society, on the other hand, people live by freely selecting a variety of life styles. In other words, people get up at different times, go to bed at different times, and eat different meals at different times. That is to say, people have living habits much different from each other. In addition, due to enhanced convenience and diversified work styles, there are a number of people each having a much reduced length of an exercise time and a much decreased intensity of the exercise.

The diversification of life styles has been making progress at a rapid pace during several past decades which can be said to be a very short period of time. Thus, a physiological adaptation to changes of the life styles cannot catch up with the changes. As a result, the number of people complaining about physical and spiritual disorders has been increasing. In such a situation, people have to take their own life styles into consideration in order to properly control their health. In addition, with the life style diversifying, there is a demand for establishment of strategies of developing and marketing products oriented to specific life styles and developing and marketing services oriented to the same.

In order to properly control health in accordance with life styles of people and establish strategies of developing and marketing every product as well as every service, which are oriented to the life styles, it is necessary to provide means for expressing and evaluating the diversified life styles in a uniform manner. However, even though there is an indicator usable for individually expressing and evaluating living habits, there is no indicator usable for comprehensively expressing and evaluating the living habits as life styles. As described above, information on forms of the living habit includes the time of getting up, the time of going to bed, the substances of the meals, the times of eating meals, the length of the exercise time and the intensity of the exercise.

By the way, living habits such as sleeping, eating and sports can each be perceived as an external factor each having an effect on a biological clock which dominates a wide variety of biological phenomena. The biological clock beats out a periodical rhythm seen in a living body as a rhythm which is vibrating independently. A circadian rhythm known as the periodical rhythm beaten out by the biological clock extensively dominates a daily-variation rhythm which is seen in biological phenomena such as the sleep-wake cycles, the body temperature, the blood pressures and the hormonal secretion. In the following description, the periodical rhythm beaten out by the biological clock is referred to as a biological-clock rhythm. In addition, as is generally known, the circadian rhythm also gets engaged with the standard of physical and mental activities, the capacity for exercises and the drug sensitivity.

The biological-clock rhythm is controlled by a gene group which is composed of clock genes. The clock genes function as a biological clock which periodically drives (or vibrates) expressions, activities, localizations and the like. The biological-clock rhythm beaten up in every individual cell by the clock genes produces a high-order biological-clock rhythm among a plurality of cells. Then, this high-order biological-clock rhythm further produces a higher-order biological rhythm inside every tissue and inside every internal organ. A biological-clock core existing at a suprachiasmatic nucleus controls these biological-clock rhythms existing between cells, inside tissues and inside internal organs, playing a role in adjustment of the rhythms to a fixed one.

PCT Patent Publication No. WO04/012128 referred to as Patent Document 1 discloses technologies such as a method for inferring a biological clock on the basis of mRNA (messenger ribonucleic acid) measurement data of a specimen material taken from a bion. In accordance with this method for inferring a biological clock, a molecular clock table for inferring a biological clock on the basis of the expression level of the mRNA is created.

Japanese Patent Laid-Open No. Hei 6-189914 referred to as Patent Document 2 hereinafter discloses a biological-rhythm curve measurement apparatus for creating a biological-rhythm curve from measured values of the deep-part body temperature of a human being. This biological-rhythm curve measurement apparatus has been designed to be capable of creating a true biological-rhythm curve by eliminating effects of external disturbances (or effects generated from external sources). By virtue of the method for inferring a biological clock and the biological-rhythm curve measurement apparatus, it is possible to infer or measure an actual biological clock (or an actual biological-clock rhythm beaten out by the actual biological clock) of a bion.

While being affected by a variety of external factors such as the times of getting up and going to bed, the substances of the meals, the times of eating meals, the length of the exercise time and the intensity of the exercise, the biological-clock rhythm is synchronized. The external factors control the biological-clock rhythm to a fixed rhythm by shifting the period of a curve representing the biological-clock rhythm in the forward or backward direction and by amplifying or attenuating the amplitude of the curve representing the biological-clock rhythm. If an external factor changes all of a sudden, the period or amplitude of the curve representing the biological-clock rhythm are also changed. A good example a phenomenon attributed to a change of the period or amplitude of the curve representing the biological-clock rhythm is a jet lag syndrome caused by an abrupt change of the times of getting up and going to bed (“Jet lag: trends and coping strategies.” Lancet 2007, 369 (9567): 1117-29, referred to as Non-Patent Document 1 hereinafter). In addition, evidences have indicated that a change of the biological-clock rhythm can serve as a cause of an attack in a patient suffering from a disease such as cancer, diabetes, a vascular disease or a neurodegenerative disease. In recent years, it is also pointed out that a change of the biological-clock rhythm contributes to an attack in a patient suffering from a mental disorder such as a psychiatric disease or a depression.

### SUMMARY

As described above, in order to properly control health in accordance with life styles of individual persons and establish strategies of developing and marketing every product and every service, which are oriented to the life styles, it is necessary to provide means for expressing and evaluating the diversified life styles in a uniform manner. It is a main desire of the present application to provide such means.

In order to solve the problems described above, the present embodiment provides a life-style evaluation method including the steps of acquiring information on a form of a living habit prescribing a life style, obtaining a virtual biological-clock rhythm curve by reflecting the information in a reference biological clock rhythm curve on a basis of the degree of an effect given by the form of the living habit on a biological-clock rhythm curve, and setting an indicator of the life style on a basis of the virtual biological-clock rhythm curve.

In accordance with this life-style evaluation method, a life style is evaluated on the basis of a difference in phase between the reference biological-clock rhythm curve and the virtual biological-clock rhythm curve.

In the life-style evaluation method, the reference biological-clock rhythm curve can be expressed by Eqs. (1) and (2) given below. Also, the virtual biological-clock rhythm curve can be expressed by Eqs. (3) to (6) given below.

$$\begin{matrix}A ( t ) = \int F ( θ ) P ( t , θ ) d θ = \int sin ( 2 π θ ) P ( t , θ ) d θ & ( 1 ) \\ P ( t , θ ) = {\sum }_{keZ} \frac{1}{\sqrt{2 π {σ}^{2}}} exp ( \frac{- {( θ - w t - {θ}_{0} + k )}^{2}}{2 {σ}^{2}} ) & ( 2 ) \\ \overline{A} ( t ) = \int F ( \overline{θ} ) \overline{P} ( t , \overline{θ} ) d \overline{θ} & ( 3 ) \\ \overline{θ} = PTC ( θ ) & ( 4 ) \\ PTC ( θ , α ) = θ + PRC ( θ , α ) & ( 5 ) \\ PRC ( θ , α ) = arg ( exp [ 2 π t θ ] + t α ) / 2 π - θ & ( 6 )\end{matrix}$$

In the equations given above, symbol t denotes time, symbol ω denotes the period of the reference biological-clock rhythm curve, symbol θ denotes the phase of the biological-clock rhythm curve, symbol δ denotes a variation of the phase and symbol α denotes the degree of an effect given by a form of the living habit on the reference biological-clock rhythm curve.

In addition, the present embodiment also provides an information processing apparatus having a first section configured to acquire information on a form of a living habit prescribing a life style, and a second section configured to compute a virtual biological-clock rhythm curve by reflecting the information in a reference biological clock rhythm curve on a basis of the degree of an effect given by the form of the living habit on a biological-clock rhythm curve.

This information processing apparatus can be provided with a section configured to compute a difference in phase between the reference biological-clock rhythm curve and the virtual biological-clock rhythm curve and a section configured to acquire the information on a form of the living habit prescribing the life style.

In this information processing apparatus, the section configured to acquire the information on a form of a living habit includes at least one of an acceleration sensor, a cardiac-beat sensor and a photo sensor.

In addition, the present embodiment also provides a life-style evaluation program having at least the step of computing a virtual biological-clock rhythm curve by reflecting information on a form of the living habit prescribing a life style in a reference biological-clock rhythm curve on a basis of the degree of an effect given by the form of the living habit on a biological-clock rhythm curve. On top of that, the present embodiment also provides a recording medium used for recording a life-style evaluation program in a form readable by a computer as a program which includes at least the step of computing a virtual biological-clock rhythm curve by reflecting information on a form of a living habit prescribing a life style in a reference biological-clock rhythm curve on a basis of the degree of an effect given by the form of the living habit on a biological-clock rhythm curve.

In the present embodiment, the living habit includes a wide variety of activities carried out during a day-to-day life as day-to-day activities such as sleeping, eating a meal, doing an exercise, taking a bath, drinking, smoking, going to work, going to school and watching a TV or a monitor. In one aspect, these activities can be perceived as physical and mental stress loads borne in doing a day-to-day life. Thus, the living habit of the present embodiment can be perceived in the same way as a variety of stresses (or a variety of stress generators) in the day-to-day life. The life style means the implementation of the day-to-day life prescribed by forms of the living habit, the information on which includes the times of going to bed and getting up in the sleeping activity, the substance of every meal in the meal eating activity, the time of eating a meal in the meal eating activity, the length of the exercise time in the exercise activity and the intensity of the exercise in the exercise activity. The life style can be perceived as a variety of stress intensities in the day-to-day life.

The biological-clock rhythm curve in the present embodiment is a curve expressing a periodical rhythm beaten out by the biological clock. The biological-clock rhythm curve is a sinusoidal curve (or a cosine curve) which has a constant phase, a constant period and a constant amplitude. For more information, the reader is suggested to refer also to Patent Document 2. To put it more concretely, the biological-clock rhythm curve can be expressed by Eqs. (1) and (2) given before. As described previously, in these equations, symbol t denotes time, symbol ω denotes the period of the reference clock rhythm curve, symbol θ denotes the phase of the biological-clock rhythm curve, symbol δ denotes a variation of the phase and symbol α denotes the degree of an effect given by a form of the living habit on the reference biological-clock rhythm curve.

Symbol δ denoting a variation of the phase and symbol α denoting the degree of such an effect are explained more as follow. As described before, the biological-clock rhythm is produced by gathering rhythms, which have been created by clock genes inside individual cells, between adjacent cells and further gathering the rhythms gathered between adjacent cells in tissues and internal organs. Eqs. (1) and (2) express the biological-clock rhythm as a set of such rhythms existing between individual cells, inside tissues and inside organs.

While being synchronized by a biological-clock core existing at a suprachiasmatic nucleus, these rhythms existing between individual cells, inside tissues and inside organs beat out their own rhythms, and phase shifts are generated between the generated rhythms. Thus, the biological-clock rhythm serving as the set of rhythms existing between individual cells, inside tissues and inside organs has a phase, a period and an amplitude which exhibit variations reflecting the phase shifts of the rhythms included in the set to serve as the biological-clock rhythms existing between individual cells, inside tissues and inside organs. The smaller the phase shifts of the rhythms included in the set to serve as the biological-clock rhythms existing between individual cells, the larger the amplitude of the biological-clock rhythm obtained by gathering the rhythms included in the set. The biological-clock rhythm having a large amplitude forms the so-called regular rhythm. Due to effects of external factors, the phase shifts of the rhythms existing between individual cells, inside tissues and inside organs increase or decrease. With the phase shifts of the rhythms of individual cells, issues and internal organs increasing or decreasing, the phase, period and amplitude of the biological-clock rhythm serving as the set of rhythms existing between individual cells, inside tissues and inside organs change. If the phase shifts of the rhythms existing between individual cells, inside tissues and inside organs increase due to effects of external factors, the biological-clock rhythm typically enters an out-of-synchronization state which is typically seen in a jet lag syndrome for example. In the out-of-synchronization state, the amplitude of the biological-clock rhythm serving as the set of rhythms existing between individual cells, inside tissues and inside organs decreases. In the present embodiment, symbol δ denotes the phase shift of the rhythms existing between individual cells, inside tissues and inside organs.

Symbol α denoting the degree of an effect given by a form of the living habit on the reference biological-clock rhythm curve is defined as a parameter prescribing the magnitudes of changes caused by effects given by external factors on the biological clock as the changes of the phase, period and amplitude which each serve as a quantity related the biological-clock rhythm serving as the set of rhythms existing between individual cells, inside tissues and inside organs. With a variety of external factors giving effects on the rhythms existing between individual cells, inside tissues and inside organs, the period of the biological-clock rhythm is shifted in the forward or backward direction and the amplitude of the biological-clock rhythm is amplified or attenuated. The magnitudes of the changes of the period and the amplitude can be prescribed by parameters each representing the type and intensity of one of the external factors. Typical examples of the external factors are forms of the living habit, information on which includes the times of going to bed and getting up, the substance of every meal, the time of eating a meal, the length of the exercise time and the intensity of the exercise.

In order to properly control health in accordance with life styles of individual persons and establish strategies of developing and marketing every product and every service, which are oriented to the life styles, the present embodiment provides means for expressing and evaluating the diversified life styles in a uniform manner.

Additional features and advantages are described herein, and will be apparent from the following Detailed Description and the figures.

### BRIEF DESCRIPTION OF THE FIGURES

FIG. 1 is an explanatory diagram of the concept of a life-style evaluation method according to the present embodiment;

FIG. 2 is an explanatory diagram showing a reference biological-clock rhythm curve and an external-factor-affected rhythm curve;

FIG. 3 is an explanatory diagram of a typical configuration of an information processing apparatus according to the present embodiment;

FIG. 4 shows an explanatory flowchart representing operations carried out by the information processing apparatus according to the present embodiment; and

FIG. 5 is an explanatory diagram showing a typical display of the reference biological-clock rhythm curve and a virtual biological-clock rhythm curve on a monitor employed in the information processing apparatus according to the present embodiment.

### DETAILED DESCRIPTION

The present application is explained below by referring to figures according to an embodiment. It is to be noted that the embodiment described below is a typical example of a representative implementation of the present application and should not thus be interpreted as a limitation to narrow the range of the present application. It is also worth noting that the embodiment is explained in chapters which are arranged as follows.

1. Life-Style Evaluation Method

(1) Overview

(2) Computation of a Virtual Biological-Clock Rhythm Curve

(3) Evaluation of Life Styles

2. Information Processing Apparatus for Evaluating Life Styles

(1) Configuration of the Information Processing Apparatus

(2) Operations of the Information Processing Apparatus

(2-1) Acquisition of Information on Forms of the Living Habit

(2-2) Computation of a Virtual Biological-Clock Rhythm Curve

(2-3) Computation of a Phase Difference between a Reference Biological-Clock Rhythm Curve and a Virtual Biological-Clock Rhythm Curve

(2-4) Prediction of a Virtual Biological-Clock Rhythm Curve and a Phase Difference

3. Life-Style Evaluation Program

1. Life-Style Evaluation Method

(1) Overview

All living habits prescribing a life style give an effect on a biological clock and can be perceived as external factors which synchronize (or change) a biological-clock rhythm beaten out by the biological clock. The living habits include sleeping, eating meals and doing exercises. Inventors of the present application have innovated a single indicator for measuring a variety of living habits through the biological clock.

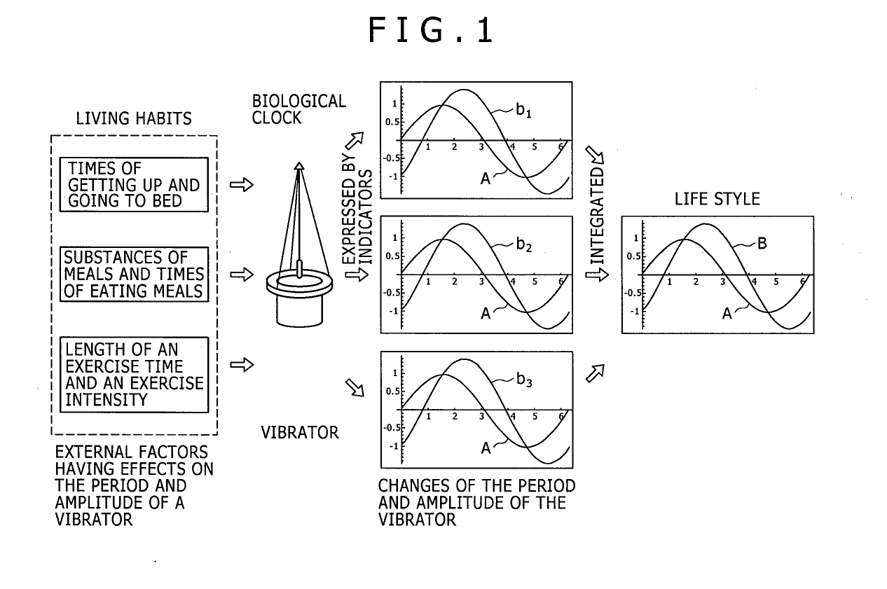

First of all, as shown in a diagram of FIG. 1, the inventors of the present application perceived the biological clock as an independently vibrating vibrator and perceived the living habits as external factors which have an effect on vibrator quantities such as the period and amplitude of the vibrator. Then, the inventors of the present application made an attempt to measure the forms of the living habit in terms of magnitudes of changes brought about by the living habits to the vibrator quantities such as the period and amplitude of the vibrator. This vibrator beats out a biological-clock rhythm of the entire human body. The biological-clock rhythm is an average of rhythms which are gathered from locations between individual cells, inside tissues and inside organs.

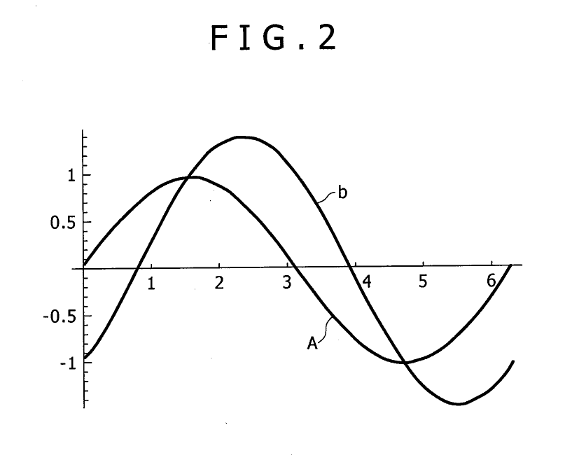

A curve representing the biological-clock rhythm beaten out by the independently vibrating vibrator is a sinusoidal curve (or a cosine curve) which has a constant phase, a constant period and a constant amplitude. The sinusoidal curve is shown as a curve denoted by symbol A in the diagram of FIG. 2. In the diagram of FIG. 2, the horizontal axis represents the lapse of time whereas the vertical axis represents the amplitude of the sinusoidal curve A. In the following description, the sinusoidal curve A is referred to as a reference biological-clock rhythm curve A which is a biological-clock rhythm curve used as a reference.

When a living habit serving as an external factor gives an effect on the vibrator, the vibrator quantities such as the period and amplitude of the vibrator change, varying the rhythm quantities such as the period and amplitude of the biological-clock rhythm beaten out by the vibrator. Thus, for example, the curve representing biological-clock rhythm changes to a sinusoidal curve denoted by symbol b in the diagram of FIG. 2. In the following description, the sinusoidal curve denoted by symbol b is referred to as an external-factor-affected rhythm curve b.

For example, the time of getting up and the time of going to bed serve as external factors which have an effect on the biological-clock rhythm, changing the reference biological-clock rhythm curve A to an external-factor-affected rhythm curve bₗ as shown in the diagram of FIG. 1. The magnitudes of changes from the quantities related to the reference biological-clock rhythm curve A to the quantities related to the external-factor-affected rhythm curve b₁ correlate to the times of getting up and going to bed. The changing quantities include the period and amplitude of the rhythm curve. In the following description, each of the magnitudes of changes in quantity is referred to simply as a quantity change magnitude. Let us take the jet lag syndrome described above as an example. In this case, if the times of getting up and going to bed are regular, the quantity change magnitudes are small. If the times of getting up and going to bed are irregular, the quantity change magnitudes increase to a certain extent. Thus, the magnitudes of the changes from the quantities related to the reference biological-clock rhythm curve A to the quantities related to the external-factor-affected rhythm curve b₁ can be used as an indicator representing the times of getting up and going to bed.

By the same token, the substance of a meal and the time of eating a meal serve as external factors, changing the reference biological-clock rhythm curve A to an external-factor-affected rhythm curve b₂ as shown in the diagram of FIG. 1. Thus, the magnitudes of the changes from the quantities related to the reference biological-clock rhythm curve A to the quantities related to the external-factor-affected rhythm curve b₂ can be used as an indicator representing the substance of a meal and the time of eating a meal. In the same way, the length of an exercise time and the intensity of an exercise serve as external factors which have an effect on the biological-clock rhythm, changing the reference biological-clock rhythm curve A to an external-factor-affected rhythm curve b₃ as shown in the diagram of FIG. 1. Thus, the magnitudes of the changes from the quantities related to the reference biological-clock rhythm curve A to the quantities related to the external-factor-affected rhythm curve b₃ can be used as an indicator representing the length of an exercise time and the intensity of an exercise.

As described above, the magnitudes of the changes from the quantities related to the reference biological-clock rhythm curve A to the quantities related to the external-factor-affected rhythm curves b₁ to b₃ can each be used as an individual indicator which represents forms of the living habit. As shown in the diagram of FIG. 1, the individual indicators each representing forms of the living habit can be integrated into a single indicator compatible with the individual indicators. That is to say, as shown in the diagram of FIG. 1, the external-factor-affected rhythm curves b₁ to b₃ are integrated into a virtual biological-clock rhythm curve denoted by symbol B. In the following description, a single rhythm curve obtained as a result of integrating external-factor-affected rhythm curves is referred to as a virtual biological-clock rhythm curve B.

The external-factor-affected rhythm curves bₗ to b₃ sustain information on the forms of the living habit prescribing the life style as changes from the quantities related to the reference biological-clock rhythm curve A to the quantities related to the external-factor-affected rhythm curves b₁, b₂ and b₃. Thus, the virtual biological-clock rhythm curve B obtained as a result of integrating the external-factor-affected rhythm curves b₁ to b₃ can be perceived certainly as a sustainer of information on the life style. For this reason, in the life-style evaluation method according to the present embodiment, the virtual biological-clock rhythm curve B is used as an indicator for evaluating the life style of each individual person.

(2) Computation of a Virtual Biological-Clock Rhythm Curve

There is a report indicating that, in accordance with results of an analysis conducted on changes of the phase of a biological clock affected by light serving as an external factor, the reference biological-clock rhythm curve A and the external-factor-affected rhythm curve b can be expressed by a model by making use of Eqs. (1) to (6) given previously (referred to “Melanopsin-dependent photo-perturbation reveals de-synchronization underlying the singularity of mammalian circadian clocks.” Nature Cell Biology, 2007, 9 (11): 1327-34).

Eqs. (1) and (2) listed below express the reference biological-clock rhythm curve A. In these equations, symbol t denotes time, symbol ω denotes the period of the reference biological-clock rhythm curve, symbol θ denotes the phase of the reference biological-clock rhythm curve and symbol δ denotes a variation of the phase.

$$\begin{matrix}A ( t ) = \int F ( θ ) P ( t , θ ) d θ = \int sin ( 2 π θ ) P ( t , θ ) d θ & ( 1 ) \\ P ( t , θ ) = {\sum }_{keZ} \frac{1}{\sqrt{2 π {σ}^{2}}} exp ( \frac{- {( θ - wt - {θ}_{0} + k )}^{2}}{2 {σ}^{2}} ) & ( 2 )\end{matrix}$$

Eqs. (3) to (6) listed below express the external-factor-affected rhythm curve b. In these equations, symbol α denotes the degree of an effect given by a form of the living habit on the reference biological-clock rhythm curve A. The effect degree α is a parameter which prescribes the magnitudes of changes caused by an external factor as the changes from quantities such as the period and amplitude of the reference biological-clock rhythm curve A to the quantities of the external-factor-affected rhythm curve b. As described above, the external factor in this typical case is light.

A̅(t)=∫F( θ̅) P̅(t, θ̅)d θ̅ (3)

θ̅=PTC(θ) (4)

PTC(θ,α)=θ+PRC(θ,α) (5)

PRC(θ,α)=arg(exp [2πtθ]+tα)/2π−θ (6)

In the same way as light serving as an external factor, for another living-habit form serving as another external factor, by reflecting information on the other form of the living habit in the reference biological-clock rhythm curve A and by making use of the effect degree α, an external-factor-affected rhythm curve b for the other form of the living habit can be obtained. As described earlier, the effect degree α prescribes the magnitudes of changes caused by the other external factor as the changes from quantities such as the period and amplitude of the reference biological-clock rhythm curve A to quantities of the external-factor-affected rhythm curve b.

Then, for a form of the living habit, the information on which includes the times of going to bed and getting up, the substance of every meal, the time of eating a meal, the length of the exercise time and the intensity of the exercise, a proper parameter used as the effect degree α is selected. Subsequently, an external-factor-affected rhythm curve b for each of the forms of the living habit is obtained by making use of the effect degree α. Then, the external-factor-affected rhythm curves b each obtained for one of the forms of the living habit are integrated to give a virtual biological-clock rhythm curve B. As a result, the virtual biological-clock rhythm curve B can also be expressed by a model by making use of Eqs. (3) to (6). In this case, the effect degree a for a form of the living habit is a parameter properly selected in accordance with the form of the living habit.

(3) Evaluation of Life Styles

The virtual biological-clock rhythm curve B obtained as a result of integrating external-factor-affected rhythm curves b as described above holds information on a plurality of forms of the living habit prescribing the life style as the magnitudes of changes from quantities pertaining to the reference biological-clock rhythm curve A to the quantities pertaining to the external-factor-affected rhythm curves b. The information on a plurality of forms of the living habit prescribing the life style is information on the life style itself. Thus, if based on these magnitudes of changes of quantities, the life styles of different persons can be evaluated by making use of a uniform indicator. In the case of this present embodiment, these magnitudes of changes of quantities are represented by a difference in phase between the reference biological-clock rhythm curve A and the virtual biological-clock rhythm curve B.

As a matter of fact, the information on the forms of the living habit is also reflected in the virtual biological-clock rhythm curve B from time to time by making use of a selected proper effect degree α as described above. Thus, the virtual biological-clock rhythm curves B can also be used as a log for recording life styles of different persons. It is expected that the log can be used as data for an analysis conducted on a correlation between a patient and the life style of the patient and data alternative to results of a diagnostic process carried out by conducting an interview with a patient about external factors such as the length of the sleeping time, the times of eating meals and the length of the exercise time in a medical examination.

2. Information Processing Apparatus for Evaluating Life Styles

(1) Configuration of the Information Processing Apparatus

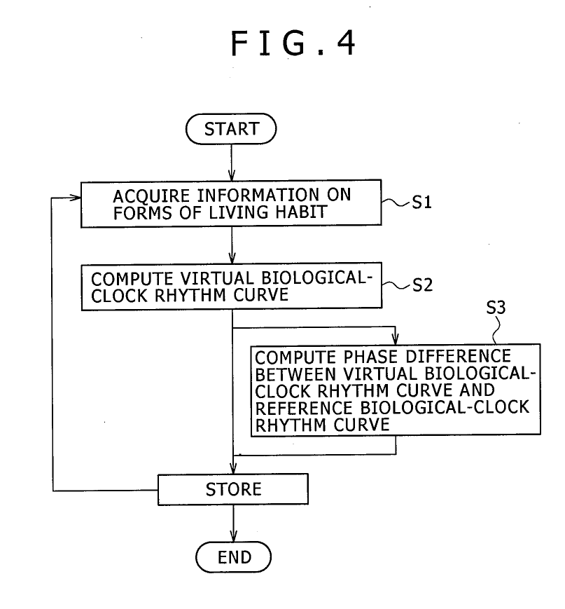

An information processing apparatus provided by the present embodiment includes means for computing a virtual biological-clock rhythm curve by reflecting information on a form of the living habit prescribing the life style in a reference biological-clock rhythm curve and by making use of the degree of an effect given by the form of the living habit on the reference biological-clock rhythm curve. A typical example of the means for computing a virtual biological-clock rhythm curve is a step S2 of a flowchart shown in FIG. 4. In addition, the information processing apparatus is also provided with means for computing a difference in phase between the reference biological-clock rhythm curve and the virtual biological-clock rhythm curve. A typical example of the means for computing the phase difference is a step S3 of the flowchart shown in FIG. 4. On top of that, the information processing apparatus also has means for acquiring information on a form of the living habit prescribing the life style. A typical example of the means for acquiring information on a form of the living habit is a step S1 of the flowchart shown in FIG. 4.

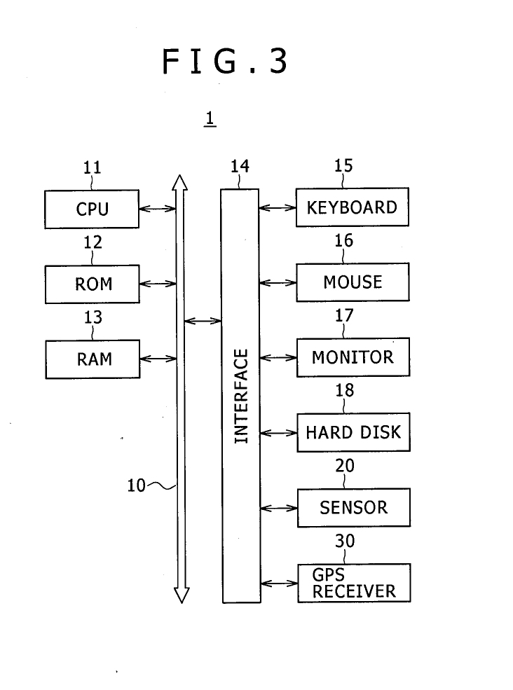

FIG. 3 is an explanatory block diagram showing the information processing apparatus 1 provided by the present embodiment. The information processing apparatus 1 is configured to employ an internal bus 10 which is typically a PCI (Peripheral Component Interconnect) or a local bus. The internal bus 10 is used for connecting a CPU 11, a ROM 12, a RAM 13 and an input/output interface 14 to each other. The CPU 11, the ROM 12, the RAM 13 and the input/output interface 14 exchange data with each other through the internal bus 10. The CPU 11 executes a program stored in advance in the ROM 12 in order to carry out processing. The RAM 13 is a memory used for storing information such as programs and data required by the CPU 11 in executions of various kinds of processing. The input/output interface 14 is connected to a keyboard 15 and a mouse 16 which are to be operated by a user in order to enter inputs to the information processing apparatus 1. The input/output interface 14 supplies operation signals to the CPU 11. Each of the operation signals is a signal which represents an input entered by the user to the information processing apparatus 1. In addition, the input/output interface 14 is also connected to a monitor 17 and a hard disc 18. The monitor 17 is a section for displaying an image in accordance with control executed by the CPU 11. The CPU 11 stores information such as a program or data into the hard disc 18 by way of the input/output interface 14 and reads out such information from the hard disc 18 by way of the input/output interface 14.

In the explanatory block diagram of FIG. 3, reference numeral 20 denotes a sensor unit which functions as a section configured to acquire information on a form of the living habit prescribing the life style. Depending on the information to be acquired, the sensor unit 20 can be configured to function as an acceleration sensor, a cardiac-beat sensor, a photo sensor and a variety of other sensors. In the explanatory block diagram of FIG. 3, reference numeral 30 denotes a GPS (Global Positioning System) receiver for acquiring positional information. Each of the sensor unit 20 and the GPS receiver 30 can be configured as a section embedded in the information processing apparatus 1 or a section external to the information processing apparatus 1.

The information processing apparatus 1 can be designed as a dedicated apparatus having the configuration shown in the explanatory block diagram of FIG. 3. As an alternative, the information processing apparatus 1 can be designed as an apparatus such as a wristwatch, a cellular phone, an AV apparatus or a personal computer. As will be described later, in order to continuously acquire information on a form of the living habit prescribing the life style from the sensor unit 20, it is desirable to design the information processing apparatus 1 as an apparatus attached to the body of the user or an apparatus always carried by the user in a state of being ready to be utilized by the user. A typical example of the apparatus attached to the body of the user is a wristwatch whereas typical examples of the apparatus always carried by the user are a cellular phone and a portable personal computer.

(2) Operations of the Information Processing Apparatus

Next, operations carried out by the information processing apparatus 1 provided by the present embodiment are explained by referring to the flowchart shown in FIG. 4 as follows.

(2-1) Acquisition of Information on Forms of the Living Habit

As shown in the figure, the flowchart begins with a step S1 at which the CPU 11 acquires information on forms of the living habit from the sensor unit 20 by way of the input/output interface 14. The information on the forms of the living habit includes the times of getting up and going to bed, the times of eating meals, the substances of the meals, the length of the exercise time and the intensity of the exercise.

It is desirable to configure the sensor unit 20 to include three sensors, i.e., an acceleration sensor, a cardiac-beat sensor and a photo sensor. The acceleration sensor is a sensor for detecting a movement of the body of the user making use of the information processing apparatus 1. The cardiac-beat sensor is a sensor for measuring the heart rate of the user. The photo sensor is a sensor for measuring the quantity of light in an environment surrounding the information processing apparatus 1 and the user. It is to be noted that the cardiac-beat sensor includes a sensor referred to as an electrocardiographic monitor or a pulse-beat sensor.

By combining the acceleration sensor, the cardiac-beat sensor and the photo sensor, it is possible to acquire information like one shown in Table 1 as the information on forms of the living habit of the user. In the table, symbol LF is an abbreviation of the words “low frequency” whereas symbol HF is an abbreviation of the words “high frequency.” Each of the LF and the HF is a numerical value showing a cardiac-beat variation (or a cardiac-beat fluctuation) which serves as an indicator of the autonomic nerve activity. An R wave of an electrocardiogram generated by the cardiac-beat sensor and an R-wave interval (also referred to as an R-R interval) between two consecutive R waves change periodically. The period of the periodical change of the R-R interval is subjected to a frequency analysis. A result of the frequency analysis indicates that a peak appears in close proximity to frequencies of 0.1 Hz and 0.25 Hz. Frequencies in close proximity to 0.1 Hz are referred to as the LF whereas frequencies in close proximity to 0.25 Hz are referred to as the HF. The ratio LF/HF is interpreted as the indicator of a sympathetic nerve activity whereas the HF is interpreted as the indicator of a sub-sympathetic nerve activity.

TABLE 1

| Information on | Acceleration | Cardiac-beat sensor | | | Photo sensor |
|---|---|---|---|---|---|
| a form of the | sensor | | Cardiac-beat change | | Light |
| living habit | Body movement | Heart rate | LF/HF | HF | quantity |
| Time of getting | Increase | Increase | Increase | Decrease | Increase |
| up | | | | | |
| Time of going | Decrease | Decrease | Decrease | Increase | Decrease |
| to bed | | | | | |
| Time of starting | — | Increase | Decrease | Increase | — |
| to eat a meal | | | | | |
| Time of | — | Decrease | Increase | Decrease | — |
| finishing eating | | | | | |
| a meal | | | | | |
| Exercise | Increase | Increase | Increase | Decrease | — |
| | considerably | considerably | | | |

Table 1 is explained in detail as follows. When a body movement detected by the acceleration sensor and a heart rate measured by the cardiac-beat sensor increase and the light quantity measured by the photo sensor rises, on the basis of information received from the sensor unit 20, the CPU 11 acquires a time of getting up. When the body movement detected by the acceleration sensor and the heart rate measured by the cardiac-beat sensor decrease and the light quantity measured by the photo sensor decreases, on the basis of information received from the sensor unit 20, the CPU 11 acquires a time of going to bed.

When the body movement detected by the acceleration sensor does not change much, the heart rate measured by the cardiac-beat sensor increases, the ratio LF/HF decreases and the HF rises, on the basis of information received from the sensor unit 20, the CPU 11 acquires a time of starting to eat a meal. When the heart rate measured by the cardiac-beat sensor decreases, the ratio LF/HF increases and the HF decreases, on the other hand, on the basis of information received from the sensor unit 20, the CPU 11 acquires a time of finishing eating a meal.

When the body movement detected by the acceleration sensor and the heart rate measured by the cardiac-beat sensor increase considerably, on the basis of information received from the sensor unit 20, the CPU 11 acquires the length of an exercise time. In addition, the CPU 11 acquires the degrees, to which the body movement detected by the acceleration sensor and the heart rate measured by the cardiac-beat sensor increase considerably, as information on the intensity of an exercise.

The sensor unit 20 is also provided with a brain-wave sensor for measuring brain waves of the user and making use of the result of the measurement as information on sleeping. The information on sleeping is used in conjunction with the time of getting up and the time of going to bed to produce information on the quality of sleeping. In general, as is commonly known, brain waves including many frequencies in low ranges indicate deep sleeping and, the larger the number of such frequencies, the deeper the sleeping or the better the quality of the sleeping state. In addition, the sensor unit 20 is also provided with a blood glucose level sensor and a serum lipid concentration level sensor which are used for measuring the blood glucose level and serum lipid concentration level of the user as information on a meal. The information on a meal is used in conjunction with the time of starting to eat the meal and information the time of finishing eating the meal to produce information on the substance of the meal. In addition, the sensor unit 20 may also be conceivably provided with a thermometer for measuring the body temperature of the user and an ambient temperature.

While the user is making use of the information processing apparatus 1, the information on the forms of the living habit is continuously acquired by the CPU 11 from the sensor unit 20 which employs the acceleration sensor, the cardiac-beat sensor, the photo sensor and the other sensors. In order to continuously acquire information on the forms of the living habit prescribing the life style from the sensor unit 20, it is desirable to design the information processing apparatus 1 as an apparatus attached to the body of the user or an apparatus always carried by the user in a state of being ready to be utilized by the user. A typical example of the apparatus attached to the body of the user is a wristwatch, and typical examples of the apparatus always carried by the user are a cellular phone and a portable personal computer.

(2-2) Computation of a Virtual Biological-Clock Rhythm Curve

Then, at the next step S2, the CPU 11 reflects the acquired information on the forms of the living habit in a reference biological-clock rhythm curve A expressed by Eqs. (1) and (2) given below in order to compute a virtual biological-clock rhythm curve B modeled by making use of Eqs. (3) to (6) also given below.

$$\begin{matrix}A ( t ) = \int F ( θ ) P ( t , θ ) d θ = \int sin ( 2 π θ ) P ( t , θ ) d θ & ( 1 ) \\ P ( t , θ ) = {\sum }_{keZ} \frac{1}{\sqrt{2 π {σ}^{2}}} exp ( \frac{- {( θ - wt - {θ}_{0} + k )}^{2}}{2 {σ}^{2}} ) & ( 2 )\end{matrix}$$

In Eqs. (1) and (2) given above, symbol t denotes time, symbol ω denotes the period of the reference biological-clock rhythm curve, symbol θ denotes the phase of the reference biological-clock rhythm curve and symbol δ denotes a variation of the phase.

A̅(t)=∫F( θ̅) P̅(t, θ̅)d θ̅ (3)

θ̅=PTC(θ) (4)

PTC(θ,α)=θ+PRC(θ,α) (5)

PRC(θ,α)=arg(exp [2πtθ]+tα)/2π−θ (6)

In Eqs. (3) to (6) given above, symbol α denotes the degree of an effect given by a form of the living habit on the reference biological-clock rhythm curve A. The effect degree α is a parameter which prescribes the magnitudes of changes caused by an external factor as the changes from quantities such as the period and amplitude of the reference biological-clock rhythm curve A to the quantities of the virtual biological-clock rhythm curve B. As described above, the external factor in this typical case is light.

At the step S2, first of all, the CPU 11 acquires pieces of positional information of the information processing apparatus 1 and the user from the GPS receiver 30 through the input/output interface 14. Then, on the basis of the acquired pieces of positional information, the CPU 11 finds the time zone (or the sunrise and sundown times) of the location of the information processing apparatus 1, entering the found time zone (or the found sunrise and sundown times) to Eqs. (1) and (2) in order to compute a reference biological-clock rhythm curve A.

Subsequently, the CPU 11 computes a virtual biological-clock rhythm curve B, which is modeled by making use of Eqs. (3) to (6), by reflecting the acquired information on the forms of the living habit in the reference biological-clock rhythm curve A and by selecting a proper parameter representing the effect degree α in accordance with the forms of the living habit. The information on the forms of the living habit includes the times of getting up and going to bed, the times of eating meals, the substances of the meals, the length of the exercise time and the intensity of the exercise.

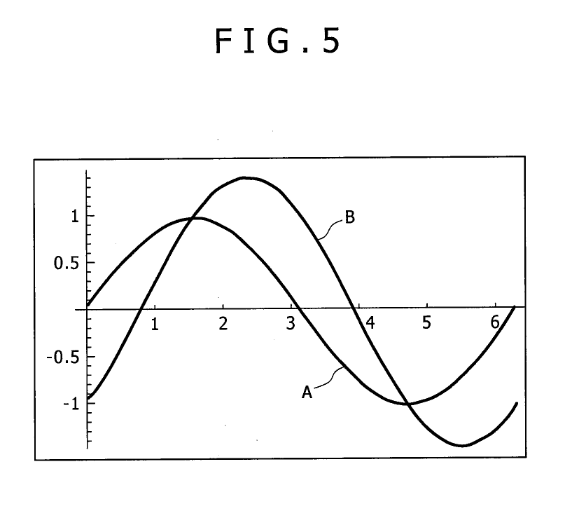

Then, the CPU 11 stores the computed reference biological-clock rhythm curve A and the computed virtual biological-clock rhythm curve B in the hard disc 18. The computed reference biological-clock rhythm curve A and the computed virtual biological-clock rhythm curve B which have been recorded in the hard disc 18 can be displayed on the monitor 17 at a request made by the user. FIG. 5 is a diagram showing a typical display of the reference biological-clock rhythm curve A and the virtual biological-clock rhythm curve B on the monitor 17.

While the user is making use of the information processing apparatus 1, the CPU 11 reflects information continuously acquired from the sensor unit 20 as information on the forms of the living habit in the reference biological-clock rhythm curve A from time to time in order to update the virtual biological-clock rhythm curve B. Thus, the virtual biological-clock rhythm curve B stored in the hard disc 18 is a curve which changes in response to changes of the forms of the living habit.

As described above, the virtual biological-clock rhythm curve B reflects information on a plurality of forms of the living habit prescribing the life style of the user. The virtual biological-clock rhythm curve B reflects such information as the magnitudes of changes from the quantities of the reference biological-clock rhythm curve A to the quantities of the virtual biological-clock rhythm curve B. As described previously, the quantities of a biological-clock rhythm curve include the period and amplitude of the biological-clock rhythm curve. Thus, the virtual biological-clock rhythm curve B holds information on the life style itself. Accordingly, the virtual biological-clock rhythm curve B stored in the hard disc 18 can be used as a log for recording the life style of the user.

If necessary, the user can display the reference biological-clock rhythm curve A and the virtual biological-clock rhythm curve B, which have been computed at the present point of time or computed in the past, on the monitor 17 in order to visually verify and evaluate the state of the life style of the user or the history of the life style (in reference to FIG. 5).

(2-3) Computation of a Phase Difference between a Reference Biological-Clock Rhythm Curve and a Virtual Biological-Clock Rhythm Curve

As described above, the virtual biological-clock rhythm curve B reflects information on a plurality of forms of the living habit prescribing the life style of the user as the magnitudes of changes from the quantities of the reference biological-clock rhythm curve A to the quantities of the virtual biological-clock rhythm curve B. Thus, the virtual biological-clock rhythm curve B holds information on the life style itself. To put it more concretely, the changes from the quantities of the reference biological-clock rhythm curve A to the quantities of the virtual biological-clock rhythm curve B are changes from the period and amplitude of the reference biological-clock rhythm curve A to the period and amplitude of the virtual biological-clock rhythm curve B. As is generally known, the changes from the period and amplitude of the reference biological-clock rhythm curve A to the period and amplitude of the virtual biological-clock rhythm curve B are a difference in phase between the reference biological-clock rhythm curve A and the virtual biological-clock rhythm curve B. After finding the virtual biological-clock rhythm curve B, at the next step S3, the CPU 11 computes the difference in phase between the reference biological-clock rhythm curve A and the virtual biological-clock rhythm curve B. It is to be noted, however, that the operation carried out at the step S3 to compute the difference in phase is not mandatory for the information processing apparatus 1 provided by the present embodiment.

While the user is making, use of the information processing apparatus 1, the CPU 11 updates the virtual biological-clock rhythm curve B from time to time on the basis of information continuously acquired from the sensor unit 20 as information on the forms of the living habit. Thus, the computed difference in phase also changes in response to changes of the forms of the living habit practiced by the user.

The CPU 11 stores the computed difference in phase in the hard disc 18. The computed phase difference which has been recorded in the hard disc 18 can be displayed on the monitor 17 at a request made by the user. By looking at the magnitude or variations of the phase differences, which have been computed at the present of time or computed in the past, stored in the hard disc 18 and displayed on the monitor 17, the user can verify the state of the life style of the user or the history of the life style as actual numerical values and can thus evaluate the life style.

(2-4) Prediction of a Virtual Biological-Clock Rhythm Curve and a Phase Difference

As described above, at the step S1, the CPU 11 acquires information on the forms of the living habit of the user from the sensor unit 20. Then, at the next step S2, the CPU 11 reflects the acquired information on the forms of the living habit in a reference biological-clock rhythm curve A in order to compute a virtual biological-clock rhythm curve B. Subsequently, at the next step S3, the CPU 11 computes the difference in phase between the reference biological-clock rhythm curve A and the virtual biological-clock rhythm curve B.

In addition, the information processing apparatus 1 is also capable of calculating the virtual biological-clock rhythm curve B and the difference in phase on the basis of information entered by the user by operating the keyboard 15 and the mouse 16 as the information on forms of the living habit of the user.

For example, it is possible to provide a configuration in which the user is allowed to directly enter information on forms of the living habit because the information is difficult to acquire from the sensor unit 20 which is configured to function as an acceleration sensor, a cardiac-beat sensor or a photo sensor. In this way, the CPU 11 is capable of acquiring information on more forms of the living habit and, on the basis of the information, the CPU 11 is capable of computing the virtual biological-clock rhythm curve B and the difference in phase between the reference biological-clock rhythm curve A and the virtual biological-clock rhythm curve B. In addition, if the user eats a meal at a time outside the period of time of making use of the information processing apparatus 1, the time of starting to eat the meal and the time of finishing eating the meal cannot be acquired from the sensor unit 20. In this case, the user is allowed to enter the time of starting to eat the meal and the time of finishing eating the meal after the meal eating activity so that the CPU 11 is capable of computing the virtual biological-clock rhythm curve B and the difference in phase between the reference biological-clock rhythm curve A and the virtual biological-clock rhythm curve B with a high degree of accuracy.

On top of that, it is also possible to provide a configuration in which the information processing apparatus 1 allows the user to enter information on forms of the living habit, which is scheduled for implementation in the near future, in advance by operating the keyboard 15 and the mouse 16. As explained previously, the information on the forms of the living habit includes the times of getting up and going to bed, the times of eating meals, the substances of the meals, the length of the exercise time and the intensity of the exercise. In this case, the CPU 11 is thus capable of calculating a virtual biological-clock rhythm curve B and a phase difference, which are predicted to hold true in the future. As a result, the user can visually verify calculation results displayed on the monitor 17 as results of the calculation for the predicted reference biological-clock rhythm curve A and the predicted virtual biological-clock rhythm curve B. The user can thus know the state of a future life style. In addition, by looking at the magnitude (or variations) of a phase difference displayed on the monitor 17 as the difference in phase between the reference biological-clock rhythm curve A and the virtual biological-clock rhythm curve B, the user can know the state of a life style expressed by an actual numerical value as the future life style of the user.

By the same token, it is also possible to provide a configuration in which the information processing apparatus 1 allows the user to enter information on the state of a life style desired in the near future by operating the keyboard 15 and the mouse 16. On the basis of the state of the life style desired in the near future, the CPU 11 computes information on living-habit forms required to implement the desired life style and displays the information on the forms of the living habit on the monitor 17. As explained previously, the information on the forms of the living habit includes the times of getting up and going to bed, the times of eating meals, the substances of the meals, the length of the exercise time and the intensity of the exercise. Thus, if the user lives in accordance with the information displayed on the monitor 17 as the information on forms of the living habit, the user is capable of shaping its own life style into the desired state.

3. Life-Style Evaluation Program

A life-style evaluation program provided by the present embodiment is a program to be executed by the information processing apparatus 1 to evaluate a life style. The life-style evaluation program provided by the present embodiment is stored in the ROM 12 or the hard disc 18 in the information processing apparatus 1 and executed by the CPU 11 in order to carry out the operations which have been explained earlier by referring to the flowchart shown in FIG. 4. That is to say, the life-style evaluation program provided by the present embodiment is a program implementing at least the step of reflecting information on forms of the living habit prescribing a life style in a reference biological-clock rhythm curve A and by making use of the effect degree a of an effect given by the forms of the living habit on the reference biological-clock rhythm curve A in order to compute a virtual biological-clock rhythm curve B.

The life-style evaluation program is executed by the CPU 11 to acquire information on the forms of the living habit from the sensor unit 20 employed in the information processing apparatus 1 to be used in a process of computing the virtual biological-clock rhythm curve B. As described previously, the information on the forms of the living habit includes the times of getting up and going to bed, the times of eating meals, the substances of the meals, the length of the exercise time and the intensity of the exercise. The information to be used in the process of computing the virtual biological-clock rhythm curve B may be information entered by the user after completion of a day-to-day activity carried out by the user as information on the activity by operating the keyboard 15 and the mouse 16. As an alternative, the information to be used in the process of computing the virtual biological-clock rhythm curve B may be information entered by the user in advance prior to occurrence of such an activity by operating the keyboard 15 and the mouse 16 as information on the activity.

In addition, it is also possible to provide a configuration in which the information on the forms of the living habit is secondarily acquired in an indirect manner to be used in the process of computing the virtual biological-clock rhythm curve B from a schedule management program which has been stored in the ROM 12 or the hard disc 18 along with the life-style evaluation program. The schedule management program can be a general-purpose program which is used for inputting information on social behaviors of the user. The information on social behaviors includes the times of getting up and going to bed, the time of going to work and going off work, a period of time spent on a trip, meeting times and a time of having an after-hours business contact such as an entertainment dinner with a customer or a supplier. The user enters the information on social behaviors to the schedule management program of the information processing apparatus 1 by operating the keyboard 15 and the mouse 16. The life-style evaluation program acquires information on the forms of the living habit by extracting the information on the forms of living habits from information entered by the user to the schedule management program as the information on social behaviors. As explained before, the information on the forms of living habits includes the times of getting up and going to bed, the times of eating meals, the substances of the meals, the length of the exercise time and the intensity of the exercise. The information on the forms of the living habit is then used in the execution of the process of computing the virtual biological-clock rhythm curve B.

The life-style evaluation program is presented to the user by giving the user a recording medium used for storing the program in a form that can be read out by a computer for execution. Typical examples of the recording medium used for storing the life-style evaluation program are a magnetic disc, a CD-ROM and a solid-state memory. In addition, the user can also acquire the life-style evaluation program by downloading the program from a program providing server or the like by way of communication media such as a network or a satellite.

As described so far, in accordance with the life-style evaluation method, the information processing apparatus and the life-style evaluation program which are provided by the present embodiment, the virtual biological-clock rhythm curve B is used as an indicator for evaluating life styles in a uniform manner. In addition, in accordance with the present embodiment, the state of a future life style can be calculated and predicted. On top of that, it is possible to know living-habit forms that are required for implementing the state of a desired life style.

Thus, the present embodiment is useful for evaluating the life style of the user and shaping the life style into a proper shape in order to carry out management of health for the user. To put it more concretely, for example, a life-style evaluation reference that can be intuitively understood with ease can be given to a patient whose disease condition can be improved by proper management of the life style of the patient. Thus, it is possible to support the life-style management in the long run. Typical examples of such a patient are patients of diabetes, brain infarct and a kidney disease. In addition, it is possible to support the life-style management of a healthy person in order to prevent these diseases.

On top of that, the present embodiment is also useful for designing the life style of the user in a proactive manner in order to avoid a jet lag syndrome and establish a condition in which learning and working can be done with a high degree of efficiency. In addition, the present embodiment can be applied to development of commodities and the like which are suitable for a consumer layer extracted from consumers as a layer of consumers each having a specific life style.

In accordance with the life-style evaluation method, the information processing apparatus and the life-style evaluation program which are provided by the present embodiment, it is possible to offer an indicator for evaluating life styles in a uniform manner. The present embodiment also contributes to promotions of the management of health, developments of commodities, entertainments and communications.

The present application claims priority cite in FIG. 1 Japanese Priority Patent Application JP 2009-000545 filed in the Japan Patent Office on Jan. 6, 2009, the entire content of which is hereby incorporated by reference.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A life-style evaluation method comprising:
acquiring information on a form of a living habit prescribing a life style, obtaining a virtual biological-clock rhythm curve by reflecting said information in a reference biological clock rhythm curve on a basis of the degree of an effect given by said form of the living habit on a biological-clock rhythm curve; and
setting an indicator of said life style on a basis of said virtual biological-clock rhythm curve.

2. The life-style evaluation method according to claim 1, wherein said life style is evaluated on the basis of a difference in phase between said reference biological-clock rhythm curve and said virtual biological-clock rhythm curve.

3. The life-style evaluation method according to claim 2, wherein said reference biological-clock rhythm curve is a curve expressed by Eqs. (1) and (2) given below whereas said virtual biological-clock rhythm curve is a curve expressed by Eqs. (3) to (6) given below:
$$\begin{matrix}A ( t ) = \int F ( θ ) P ( t , θ ) d θ = \int sin ( 2 π θ ) P ( t , θ ) d θ & ( 1 ) \\ P ( t , θ ) = {\sum }_{keZ} \frac{1}{\sqrt{2 π {σ}^{2}}} exp ( \frac{- {( θ - wt - {θ}_{0} + k )}^{2}}{2 {σ}^{2}} ) & ( 2 ) \\ \overline{A} ( t ) = \int F ( \overline{θ} ) \overline{P} ( t , \overline{θ} ) d \overline{θ} & ( 3 ) \\ \overline{θ} = PTC ( θ ) & ( 4 ) \\ PTC ( θ , α ) = θ + PRC ( θ , α ) & ( 5 ) \\ PRC ( θ , α ) = arg ( exp [ 2 π t θ ] + t α ) / 2 π - θ & ( 6 )\end{matrix}$$
where symbol t denotes time, symbol ω denotes the period of said reference biological-clock rhythm curve, symbol θ denotes the phases of said biological-clock rhythm curves, symbol δ denotes a variation of said phase and symbol α denotes the degree of said effect.

4. The life-style evaluation method according to claim 3, wherein said information on a form of a living habit includes at least a time of getting up, a time of going to bed, times of eating meals, the length of an exercise time and the intensity of said exercise.

5. An information processing apparatus comprising:
means for acquiring information on a form of a living habit prescribing a life style; and
means for computing a virtual biological-clock rhythm curve by reflecting said information in a reference biological clock rhythm curve on a basis of the degree of an effect given by said form of the living habit on a biological-clock rhythm curve.

6. The information processing apparatus according to claim 5, further comprising:
means for computing a difference in phase between said reference biological-clock rhythm curve and said virtual biological-clock rhythm curve.

7. The information processing apparatus according to claim 6, further comprising:
means for acquiring said information on a form of a living habit prescribing a life style.

8. The information processing apparatus according to claim 7, wherein said means for acquiring said information on a form of a living habit includes at least one of an acceleration sensor, a cardiac-beat sensor and a photo sensor.

9. A life-style evaluation program comprising:
at least the step of computing a virtual biological-clock rhythm curve by reflecting information on a form of a living habit prescribing a life style in a reference biological-clock rhythm curve on a basis of the degree of an effect given by said form of the living habit on a biological-clock rhythm curve.

10. A recording medium used for recording a life-style evaluation program in a form readable by a computer as a program which includes at least the step of computing a virtual biological-clock rhythm curve by reflecting information on a form of a living habit prescribing a life style in a reference biological-clock rhythm curve on a basis of the degree of an effect given by said form of the living habit on a biological-clock rhythm curve.

11. An information processing apparatus comprising:
a first section configured to acquire information on a form of a living habit prescribing a life style; and
a second section configured to compute a virtual biological-clock rhythm curve by reflecting said information in a reference biological clock rhythm curve on a basis of the degree of an effect given by said form of the living habit on a biological-clock rhythm curve.
